(19) United States
(12) Patent Application Publication
(10) Pub. No.: US 2022/0110314 A1
(43) Pub. Date: Apr. 14, 2022

(54) NUCLEIC ACID PRESERVATION, PREPARATION METHOD AND APPLICATION THEREOF

(71) Applicants: Guangdong Longsee Biomedical Co. LTD., Guangzhou (CN); Foshan Langsee Biotechnology Co. LTD., Foshan (CN)

(72) Inventors: Zhao Zhang, Guangzhou (CN); Jiayin Diao, Guangzhou (CN); Yan Ma, Guangzhou (CN); Kangdi Zheng, Guangzhou (CN); Tao Chen, Guangzhou (CN)

(73) Assignees: Guangdong Longsee Biomedical Co. LTD., Guangzhou (CN); Foshan Langsee Biotechnology Co. LTD., Foshan (CN)

(21) Appl. No.: 17/105,623

(22) Filed: Nov. 26, 2020

Related U.S. Application Data
(63) Continuation of application No. PCT/CN2020/121876, filed on Oct. 19, 2020.

(30) Foreign Application Priority Data
Oct. 12, 2020 (CN) 2020110867140

(51) Int. Cl. A01N 1/02

(52) U.S. Cl. CPC A01N 1/021

(57) ABSTRACT

A nucleic acid preservation solution, a preparation method and application for same is disclosed. The nucleic acid preservation solution of the invention includes, by weight percentage, citric acid 13%-35%, Tween 20 0.50%-2.50%, disodium ethylene diamine tetraacetic acid (EDTA) 13%-40%, sodium sulfate 35%-70%, polyethylene glycol octyl phenyl ether 0.50%-2.50%, and the balance being diethylpyrocarbonate (DEPC) water.

### TECHNICAL FIELD

The disclosure relates to the field of biological technical products, and more specifically, to a nucleic acid preservation solution, its preparation method and application.

### BACKGROUND

Nucleic acid is a biological macromolecular compound synthesized from polynucleotides and widely exists in all animals and plants. Nucleic acids in cells and microorganisms can be divided into ribonucleic acids (RNA) and deoxyribonucleic acid (DNA) according to their chemical composition.

Accurate detection of nucleic acid is an important aspect in molecular biology sample analysis. High costs and high professionalism of nucleic acid detection usually separate the acquisition and detection of samples. Most of the samples to be tested require short-distance or long-distance transportation to enter the detection process. However, as a carrier of biological genetic information, the performance of nucleic acid is unstable. It is easy to cause nuclear degradation and denaturation under the influence of external physical factors such as temperature, humidity, ultraviolet rays, etc., chemical factors such as pH value, hydrolysis reaction, oxidation reaction, etc., and biological factors such as enzymatic hydrolysis and microbial infection. This requires strict restrictions on the transportation time of samples, which in turn, greatly increases the difficulty of sample storage during transportation.

At present, the main way to store samples is to directly store them at low temperature after collection. However, this method has major limitations. Firstly, the cost of cryopreservation is relatively high and requires special equipment such as liquid nitrogen tanks. Secondly, storing samples in ultra-low temperature for long-term storage would bring in the state and structure change of nucleic acids, resulting in inaccurate test results. Based on this, it is necessary to develop a preservation solution that can provide a stable environment for samples under transportation conditions, prevent cell rupture and nucleic acid degradation, so as to eliminate the adverse effects on subsequent detection.

Therefore, it is urgent for those skilled in the art to provide a nucleic acid preservation solution, a preparation method and an application thereof.

### SUMMARY

In view of this, the disclosure provides a nucleic acid preservation solution, a preparation method and an application thereof.

The nucleic acid preservation solution has the advantage of maintaining the stability of nucleic acid under normal temperature transportation conditions.

In order to achieve the above objectives, the disclosure adopts the following technical scheme:

A nucleic acid preservation solution, by weight percentage, includes citric acid 13%-35%, Tween 20 0.50%-2.50%, disodium ethylene diamine tetraacetic acid (EDTA) 13%-40%, sodium sulfate 35%-70%, polyethylene glycol octyl phenyl ether 0.50%-2.50%, and the balance being diethylpyrocarbonate (DEPC) water.

Further, the solution includes, by weight percentage, citric acid 21%, Tween 20 1.50%, disodium EDTA 35.9%, sodium sulfate 40%, polyethylene glycol octyl phenyl ether 1.50%, and the balance being DEPC water.

Further, the pH of the preservation solution of the nucleic acid is 4.5-6.

Further, a preparation method of the nucleic acid preservation solution, including the steps of:

preparing the citric acid, Tween 20, disodium EDTA, sodium sulfate and polyethylene glycol octyl phenyl ether according to the weight percentage, and dissolving in the DEPC water;

regulating the pH of the dissolved solution to 4.5-6 with a pH meter;

sterilizing the regulated solution through a 0.22 μM water filtration membrane to obtain nucleic acid preservation solution.

Further, the method includes dispensing the prepared nucleic acid preservation solution aseptic and enzyme free into sampling tubes. The short-term storage and testing temperature of the sample tube is 0-50° C.; and the long-term storage temperature of the sample tube is 4° C.

Further, the preservation and transportation temperature is 0-50° C.

Preferably, the preservation and transportation temperature is 25° C.

Further, the biological samples are human mammal blood, urine, feces, sputum, saliva and throat swab collection fluid.

Further, the application of the nucleic acid preservation solution in the preservation and transportation of biological samples, the solution is used for storing coronavirus nucleic acid.

If the reagent is acidic, the pH is regulated with NaOH while if it is alkaline, the pH is regulated with concentrated HCl.

The usage method is as follows: take the fresh sample, put it into the nucleic acid preservation solution immediately, and store it under the temperature of 4-25° C.

It can be known from the above technical solutions that, compared with the prior art, the present disclosure provides a nucleic acid preservation solution and a preparation method and application thereof. The nucleic acid preservation solution has a lower raw material cost, a smaller number of components and the preparation method is simple and fast, it reduces the production cost while more accurately controlling the amount of additives. The nucleic acid storage solution can be stably stored for 15 days under the transportation condition of 0-50° C., and does not affect subsequent sample DNA/RNA extraction and detection experiments. The preservation effect is good; the nucleic acid preservation solution can be stably preserved for 2 years under the transportation conditions of 0-50° C., does not affect the subsequent sample DNA/RNA extraction and detection experiments, and has a long storage time.

### BRIEF DESCRIPTION OF DRAWINGS

In order to explain the embodiments of the present disclosure or the technical solutions in the prior art more clearly, the following will briefly introduce the drawings that need to be used in the description of the embodiments or the prior art. Obviously, the drawings in the following description are only embodiment of the present disclosure. For those of ordinary skill in the art, other drawings can be obtained according to the provided drawings without creative work.

FIG. 1 is a diagram of RNA electrophoresis of embodiment 1 of the present disclosure;

Among them, lane 1 is the result of extracting the sample immediately without preserving solution. Lanes 2-4 are the results of extracting samples after 1 day at room temperature. Lanes 5-7 are the results of extracting samples after 3 days at room temperature. Lanes 8-10 are the extraction results after being stored at room temperature for 7 days. Lanes 11-13 are the extraction results after being stored at room temperature for 14 days.

### DETAILED DESCRIPTION OF THE EMBODIMENTS

The technical solutions in the embodiments of the present invention will be clearly and completely described below in conjunction with the accompanying drawings in the embodiments of the present disclosure. Obviously, the described embodiments are only a part of the embodiments of the present invention, rather than all the embodiments. Based on the embodiments of the present invention, all other embodiments obtained by those of ordinary skill in the art without creative work shall fall within the protection scope of the present invention.

The human immune cells used in the following examples are human immune cells self-isolated and cultured from human peripheral blood, frozen and stored, activated and cultured before use. The positive quality control used was purchased from Sun Yat-sen University Daan Gene Co., Ltd.; Novel coronavirus 2019-nCoV nucleic acid detection kit (fluorescent PCR method) (DA0931), batch number: 2020026, cryopreserved.

### Embodiment 1

Take the following components of the formula and add them to the sterilized container: 21% citric acid, 1.50% Tween 20, 35.90% disodium EDTA, 40% sodium sulfate, 1.50% polyethylene glycol octyl phenyl ether; dissolve the components in the DEPC water; regulate the pH of the dissolved solution with a pH meter (temperature range is 18-25° C.) to pH 5.0-5.5; sterilize the pH regulated solution through 0.22 μM water filter membrane.

### Embodiment 2

Take the following components of the formula and add them to the sterilized container: 13% citric acid, 0.50% Tween 20, 13% disodium EDTA, 35% sodium sulfate, 0.50% polyethylene glycol octyl phenyl ether; dissolve the components in the DEPC water; regulate the pH of the dissolved solution with a pH meter (temperature range is 18-25° C.) to pH 4.5-4.8; sterilize the pH regulated solution through 0.22 μM water filter membrane.

### Embodiment 3

Take the following components of the formula and add them to the sterilized container: 30% citric acid, 2.50% Tween 20, 15% disodium EDTA, 45% sodium sulfate, 2.50% polyethylene glycol octyl phenyl ether; dissolve the components in the DEPC water; regulate the pH of the dissolved solution with a pH meter (temperature range is 18-25° C.) to pH 5.7-6.0; sterilize the pH regulated solution through 0.22 μM water filter membrane.

The performance test of the nucleic acid preservation solution prepared in embodiments 1-3 was carried out according to the following scheme.

I. Performance Test of Preservation Solution

1. The experiment was performed according to the following 5 conditions for nucleic acid preservation and extraction:

Add 10′ human immune cells to 2 mL of saline or nucleic acid preservation solution.

(1) Extract immediately without adding preservation solution:

After adding the cells to physiological saline, immediately perform RNA extraction;

(2) Extraction after 1 day:

Add the cells to the nucleic acid storage solution, store them at 25° C. for 1 day before extracting RNA;

(3) Extract after 3 days:

Add the cells to the nucleic acid storage solution, store them at 25° C. for 3 days, and then extract RNA;

(4) Extract after 7 days:

Add the cells to the nucleic acid storage solution, and then store at 25° C. for 7 days before extracting RNA;

(5) Extraction after 14 days:

Add the cells to the nucleic acid storage solution, and then store at 25° C. for 14 days before extracting RNA.

2. RNA extraction uses the nucleic acid extraction or purification kit (viral RNA rapid extraction kit-column extraction) produced by Guangdong Nanxin Medical Technology Co., Ltd. The specific methods are as follows:

(1) Take 200 μL of the sample after adding the nucleic acid storage solution to a 1.5 mL nuclease-free centrifuge tube.

(2) Add 560 μL Buffer VL working solution, 10 μL proteinase K (20 mg/mL), and vortex for 15 s.

(3) Incubate at room temperature (15-25° C.) for 10 min; or at 70° C. for 5 min.

(4) Centrifuge briefly to collect the droplets on the tube wall.

(5) Add 560 μL of absolute ethanol (96%-100%), vortex for 15 s, and centrifuge briefly.

(6) Take 630 μL of the above mixed solution in the purification column, centrifuge at 8000 rpm for 1 min, and discard the filtrate.

(7) Repeat step 6 until the mixture is completely transferred.

(8) Add 500 μL W1 to the purification column, centrifuge at 8000 rpm for 1 min, and discard the filtrate.

(9) Add 500 μL W2 to the purification column, centrifuge at 12,000 rpm for 3 minutes, and discard the filtrate.

(10) Place the purification column in a new 2 mL nuclease-free Ep tube and centrifuge at 12,000 rpm for 1 min.

(11) Place the purification column in a new 1.5 mL nuclease-free Ep tube, open the lid, and stand at room temperature for 5 minutes to thoroughly dry the remaining rinse solution in the adsorbent.

(12) Add 50 μL of 65° C. preheated Buffer VE to the middle of the adsorption column membrane and stand at room temperature for 1 min.

(13) Centrifuge at 12,000 rpm for 2 min to elute.

(14) Perform RNA quantification.

(15) Quantitative qPCR (using the new coronavirus 2019-nCoV nucleic acid detection kit of Sun Yat-sen University Daan Gene Co., Ltd. (fluorescence PCR method), the N gene probe is labeled with FAM, and the ORF lab gene probe is labeled with VIC, the internal marker gene probe is labeled with Cy5).

II. Stability Test of Preservation Solution

1. Accelerated stability: Divide the same batch of storage solution into 5 parts and respectively place them at 4° C., 16° C., 25° C., 37° C., and 45° C. for 1 month. Add 2 mL of the experimental liquid at each temperature for 1 day, 3 days, 7 days, 14 days and 28 days to each tube containing human immune cells, and store at 25° C. for 1 day before detecting nucleic acid contained therein.

2. Real-time stability: Choose a certain number of three batches of preservation solutions and store them in a dry and cool environment at 25° C. From the date of production, it is placed 1 month after the expiration date, a total of 25 months. Add 2 mL of the preservation solution stored for 1, 2, 3, 6, 9, 12, 18, 24, and 25 months to human immune cells, and place them at 25° C. for one day before extracting nucleic acid for detection.

3. Temperature influence: Take the same batch of storage solution within the validity period under normal storage conditions and put it in the laboratory temperature and humidity environment (temperature 25±2° C., humidity 60±2% RH) for 6 hours, and place it under the temperature of 40° C. Then place in a 90% RH temperature and humidity test box for 72 hours, observe the damage of the product according to the acceptable damage limit of the product, if the product is confirmed to be damaged, the test fails; otherwise, a further pressure impact test is to be conducted.

4. Pressure influence: take the same batch of storage solution within the validity period under normal storage conditions, place it in the center of the pressure plate of the press, and uniformly pressurize at a rate of 0.5 in/min (13 mm/min) and make the pressure reach 17.57 in (Simulate low air pressure at an altitude of 4267 meters), maintain the pressure for 1 hour, observe the damage of the product according to the acceptable damage limit of the product, if the product is confirmed to be damaged, the test fails; otherwise, a further shock impact test is to be conducted.

5. Shock impact: take the same batch of preservation solution within the validity period under normal preservation conditions, choose random shock mode among the frequencies of 1 Hz, 4 Hz, 100 Hz and 200 Hz, and place the product on the shaking table for 30 minutes, turn over (that is, the top face down) and shake for 10 minutes, the front (or back) face down for 10 minutes, and the side face down for 10 minutes. After stopping the shaking, observe the damage of the product according to the acceptable damage limit of the product. If the product is confirmed to be damaged, the test fails; otherwise, a further drop impact test is to be conducted.

6. Drop impact: Take the same batch of storage solution within the validity period under normal storage conditions, place the sample at a height of 32 inches, drop the product at different angles, and observe the damage of the product according to the acceptable damage limit of the product. If the product is confirmed to be damaged, it failed the test.
Use the nucleic acid preservation solution prepared in embodiment 1 to perform a performance test on the positive quality control of the 2019 novel coronavirus, according to the following scheme:

The experiment was carried out according to the following 4 conditions for nucleic acid storage and extraction:

(1) Extract immediately without adding preservation solution: add 400 μL of positive quality control to 2 mL of saline, and immediately perform RNA extraction;

(2) Add storage solution and extract immediately: Add 400 μL of positive quality control to 2 mL of nucleic acid storage solution, and then perform RNA extraction after storing at 25° C. for 1 day;

(3) Extract after standing for 3 days: Add 400 μL of positive quality control to 2 mL of nucleic acid storage solution, and then perform RNA extraction after storing at 25° C. for 3 days.

(4) Extract after standing for 7 days: Add 400 μL of positive quality control to 2 mL of nucleic acid storage solution, and then perform RNA extraction after storing at 25° C. for 7 days.
The performance test results of the nucleic acid preservation solution prepared in Embodiment 1
(1) Human immune cells were added to the nucleic acid storage solution prepared in Embodiment 1 and stored at room temperature for 1, 3, 7 and 14 days. After RNA was extracted, the purity and concentration were determined. The experiment was repeated 3 times. The results are shown in Table 1.

TABLE 1
RNA purity and concentration

| Days of | | Purity | | Concentration | |
|---|---|---|---|---|---|
| preservation | | (A260/A280) | Mean | (ng/μL) | Mean |
| Extracting without | | 1.901 | 1.863 | 245.28 | 245.22 |
| preservative | | 1.823 | | 254.95 | |
| immediately | | 1.865 | | 235.42 | |
| 1 | day | 1.896 | 1.866 | 228.52 | 220.19 |
| | | 1.84 | | 211.4 | |
| | | 1.862 | | 220.64 | |
| 3 | days | 1.923 | 1.907 | 218.4 | 211.84 |
| | | 1.894 | | 213.68 | |
| | | 1.905 | | 203.44 | |
| 7 | days | 1.905 | 1.888 | 199.41 | 190.43 |
| | | 1.896 | | 188.67 | |
| | | 1.862 | | 183.2 | |
| 14 | days | 1.86 | 1.853 | 162.04 | 164.88 |
| | | 1.845 | | 160.16 | |
| | | 1.854 | | 172.44 | |

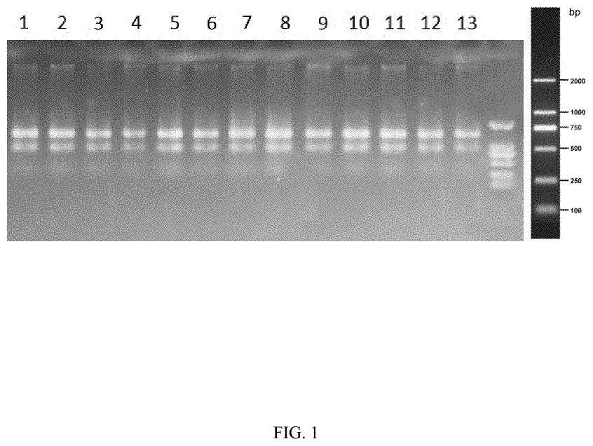

(2) Human immune cells were added to the nucleic acid storage solution prepared in embodiment 1, and RNA was extracted after being stored at room temperature for 1, 3, 7, and 14 days. The results of agarose gel electrophoresis are shown in FIG. 1.
(3) Human immune cells were added to the nucleic acid storage solution prepared in Example 1 and stored at room temperature for 1, 3, 7, and 14 days, and RNA was extracted for qPCR quantitative analysis. The experiment was repeated for 3 times, and the results are shown in Table 2.

TABLE 2
Ct value in qPCR result of RNA

| Days of | | |
|---|---|---|
| preservation | Ct Value | Mean |
| Extracting without | 18.47 | 18.56 |
| preservative | 18.56 | |
| immediately | 18.65 | |

| | | | |
|---|---|---|---|
| 1 | day | 22.48 | 22.29 |
| | | 22.14 | |
| | | 22.25 | |
| 3 | days | 22.78 | 22.60 |
| | | 22.39 | |
| | | 22.63 | |
| 7 | days | 21.52 | 20.64 |
| | | 21.77 | |
| | | 18.62 | |
| 14 | days | 22.21 | 22.46 |
| | | 22.64 | |
| | | 22.52 | |

(4) The accelerated stability results are shown in Table 3. Specifically, the nucleic acid storage solution prepared in Example 1 was placed at different temperatures and added to the RNA extracted from human immune cells, and the purity, concentration, and integrity were measured.

TABLE 3
Result of accelerated stability

| | | | | | Purity | Concen- | Number | |
|---|---|---|---|---|---|---|---|---|
| | | | | | (A260/ | tration | of bands | Clarity |
| Temperature | | Days | | pH | A280) | (ng/μL) | (28S:18S) | of band |
| Control | | no | | 5.21 | 1.836 | 145.22 | 2 | ✓ |
| 4° | C. | 1 | d | 5.18 | 1.856 | 132.42 | 2 | ✓ |
| | | 3 | d | 5.16 | 1.864 | 126.90 | 2 | ✓ |
| | | 7 | d | 5.16 | 1.952 | 135.53 | 2 | ✓ |
| | | 14 | d | 5.18 | 1.922 | 124.92 | 2 | ✓ |
| | | 28 | d | 5.17 | 1.892 | 118.60 | 2 | ✓ |
| 16° | C. | 1 | d | 5.11 | 1.921 | 128.36 | 2 | ✓ |
| | | 3 | d | 5.14 | 1.985 | 126.50 | 2 | ✓ |
| | | 7 | d | 5.33 | 1.864 | 119.34 | 2 | ✓ |
| | | 14 | d | 5.24 | 1.825 | 103.69 | 2 | ✓ |
| | | 28 | d | 5.17 | 1.904 | 98.80 | 2 | ✓ |
| 25° | C. | 1 | d | 5.31 | 1.835 | 135.69 | 2 | ✓ |
| | | 3 | d | 5.15 | 1.903 | 130.68 | 2 | ✓ |
| | | 7 | d | 5.12 | 1.985 | 128.70 | 2 | ✓ |
| | | 14 | d | 5.22 | 1.862 | 113.51 | 2 | ✓ |
| | | 28 | d | 5.15 | 1.936 | 99.32 | 2 | ✓ |
| 37° | C. | 1 | d | 5.32 | 1.952 | 127.34 | 2 | ✓ |
| | | 3 | d | 5.23 | 1.856 | 128.36 | 2 | ✓ |
| | | 7 | d | 5.13 | 1.832 | 112.48 | 2 | ✓ |
| | | 14 | d | 5.21 | 1.932 | 105.90 | 2 | ✓ |
| | | 28 | d | 5.20 | 1.875 | 100.35 | 2 | ✓ |
| 45° | C. | 1 | d | 5.24 | 1.921 | 118.37 | 2 | ✓ |

| | | | | | | |
|---|---|---|---|---|---|---|
| 3 | d | 5.28 | 1.853 | 112.58 | 2 | ✓ |
| 7 | d | 5.34 | 1.906 | 104.20 | 2 | ✓ |
| 14 | d | 5.25 | 1.892 | 95.24 | 2 | ✓ |
| 28 | d | 5.36 | 1.934 | 89.34 | 2 | ✓ |

(5) The real-time stability results of the three batches of storage solutions are shown in Table 4.1, Table 4.2, and Table 4.3. Specifically, the nucleic acid storage solution prepared in Example 1 is stored at room temperature 1, 2, 3, 6, 9, 12, 18. The purity, concentration and integrity of RNA extracted after adding human immune cells 24 and 25 months later were measured.

TABLE 4.1
The real-time stability results of the first batch

| | | | | | | | Number | |
|---|---|---|---|---|---|---|---|---|
| | | | | | Purity | Concentration | of bands | Clarity |
| Group | Set Time | | pH | Precipitation | (A260/A280) | (ng/μL) | (28S:18S) | of band |
| control | no | | 5.17 | no | 1.952 | 120.68 | 2 | ✓ |
| 25° C. | 1 | month | 5.16 | no | 1.925 | 91.08 | 2 | ✓ |
| | 2 | months | 5.21 | no | 1.891 | 102.68 | 2 | ✓ |
| | 3 | months | 5.30 | no | 1.920 | 105.28 | 2 | ✓ |
| | 6 | months | 5.24 | no | 1.865 | 124.00 | 2 | ✓ |
| | 9 | months | 5.37 | no | 1.827 | 98.20 | 2 | ✓ |
| | 12 | months | 5.13 | no | 1.896 | 122.16 | 2 | ✓ |
| | 18 | months | 5.14 | no | 1.903 | 111.35 | 2 | ✓ |
| | 24 | months | 5.22 | no | 1.905 | 95.80 | 2 | ✓ |
| | 25 | months | 5.18 | no | 1.992 | 88.41 | 2 | ✓ |

TABLE 4.2
The real-time stability results of the second batch

| | | | | | | | Number | |
|---|---|---|---|---|---|---|---|---|
| | | | | | Purity | Concentration | of bands | Clarity |
| Group | Set Time | | pH | Precipitation | (A260/A280) | (ng/μL) | (28S:18S) | of band |
| control | no | | 5.19 | no | 1.952 | 131.35 | 2 | ✓ |
| 25° C. | 1 | month | 5.36 | no | 1.865 | 128.52 | 2 | ✓ |
| | 2 | months | 5.22 | no | 1.952 | 115.20 | 2 | ✓ |
| | 3 | months | 5.32 | no | 1.832 | 105.28 | 2 | ✓ |
| | 6 | months | 5.21 | no | 1.865 | 104.05 | 2 | ✓ |
| | 9 | months | 5.33 | no | 1.827 | 98.20 | 2 | ✓ |
| | 12 | months | 5.21 | no | 1.920 | 122.16 | 2 | ✓ |
| | 18 | months | 5.22 | no | 1.903 | 111.35 | 2 | ✓ |
| | 24 | months | 5.15 | no | 1.912 | 90.50 | 2 | ✓ |
| | 25 | months | 5.24 | no | 1.992 | 87.56 | 2 | ✓ |

TABLE 4.3
The real-time stability results of the third batch

| | | | | | | | Number | |
|---|---|---|---|---|---|---|---|---|
| | | | | | Purity | Concentration | of bands | Clarity |
| Group | Set Time | | pH | Precipitation | (A260/A280) | (ng/μL) | (28S:18S) | of band |
| control | no | | 5.16 | no | 1.952 | 124.36 | 2 | ✓ |
| 25° C. | 1 | month | 5.21 | no | 1.824 | 115.61 | 2 | ✓ |
| | 2 | months | 5.30 | no | 1.872 | 110.36 | 2 | ✓ |
| | 3 | months | 5.24 | no | 1.831 | 109.51 | 2 | ✓ |
| | 6 | months | 5.37 | no | 1.865 | 101.00 | 2 | ✓ |
| | 9 | months | 5.23 | no | 1.902 | 97.20 | 2 | ✓ |
| | 12 | months | 5.24 | no | 1.896 | 95.16 | 2 | ✓ |
| | 18 | months | 5.22 | no | 1.887 | 97.35 | 2 | ✓ |
| | 24 | months | 5.18 | no | 1.905 | 90.50 | 2 | ✓ |
| | 25 | months | 5.19 | no | 1.992 | 85.13 | 2 | ✓ |

(6) The results of the temperature influence in the transportation stability are shown in Table 5. Specifically, the nucleic acid storage solution prepared in embodiment 1 is packaged at different temperatures and different temperature and humidity conditions.

TABLE 5
The results of the temperature influence
in the transportation stability

| | Temperature | Humidity | Time | | |
|---|---|---|---|---|---|
| Test order | (° C.) | (% R.H) | (hr) | pH | Breakage |
| Humiture | 25.5 | 59 | 6 | 5.23 | no |
| pretreatment | | | | | |
| (lab humiture) | | | | | |
| Humiture | 40 | 90 | 72 | 5.21 | no |
| treatment | | | | | |
| (controllable | | | | | |
| humiture) | | | | | |

(7) The results of pressure influence in transportation stability are shown in Table 6, specifically the integrity of packaging of the nucleic acid storage solution prepared in Example 1 under different pressures.

TABLE 6
The results of pressure influence in transportation stability

| | Altitude | Altitude | | Torr | | | Duration | | |
|---|---|---|---|---|---|---|---|---|---|
| Group | (m) | (ft) | pH | (mm HG) | In.HG | kPa | (min) | pH | Breakage |
| Low pressure | 4267 | 14000 | 6.08 | 446.33 | 17.57 | 59.5 | 60 | 5.22 | no |

(8) the results of shock impacts in transportation stability are shown in Table 7, Specifically, the nucleic acid storage solution prepared in Example 1 is packaged in completeness at different frequencies.

TABLE 7
The results of shock impacts in transportation stability

| | | | | Surface | |
|---|---|---|---|---|---|
| | Shock spectrum | | | touching the | |
| | | PSD | | shock table | |
| | Frequency | grade | Time | (surface | |
| Model | (Hz) | (g2/Hz) | (min) | number) | Breakage |
| Random | 1 | 0.0001 | 30 | 3 | no |
| shock | 4 | 0.01 | 10 | 1 | no |
| | 100 | 0.01 | 10 | 4 | no |
| | 200 | 0.001 | 10 | 6 | no |

(9) The results of the impact of the drop in the transportation stability are shown in Table 8. Specifically, the nucleic acid storage solution prepared in embodiment 1 is packaged in completeness in different directions.

Table 8 The results of the impact of the
drop in the transportation stability

| | Height of | | | |
|---|---|---|---|---|
| Number | drops | | | |
| of drops | (Inches) | Direction | | Breakage |
| 1 | 32 | angle | the weakest corner of the | no |
| | | | 3 sides, if not sure, test | |
| | | | the corner 2-3-5 | |
| 2 | 32 | edge | the shortest edge of the | no |
| | | | drop angle | |
| 3 | 32 | edge | the second longest edge | no |
| | | | of the drop angle | |
| 4 | 32 | edge | the longest edge of the | no |
| | | | drop angle | |
| 5 | 32 | surface | any smallest surface | no |
| 6 | 32 | surface | another smallest surface | no |
| 7 | 32 | surface | any medium surface | no |
| 8 | 32 | surface | another medium surface | no |
| 9 | 32 | surface | any largest surface | no |
| 10 | 32 | surface | another largest surface | no |

(10) The positive quality control of the 2019 novel coronavirus was added to the nucleic acid preservation solution prepared in embodiment 1 and stored at room temperature for 3 or 7 days, and RNA was extracted for qPCR quantitative analysis. The experiment was repeated for 3 times, and the results are shown in Table 9.

TABLE 9
Ct value of qPCR results of novel crown
virus RNA in positive quality control

| Days of | FAM Ct | | VIC Ct | | Cy5 Ct | |
|---|---|---|---|---|---|---|
| preservation | value | Mean | value | Mean | Value | Mean |
| Extracting | 31.52 | 31.67 | 29.04 | 29.38 | 27.91 | 28.10 |
| without | 32.06 | | 29.32 | | 28.34 | |
| preservative | 31.43 | | 29.79 | | 28.05 | |
| immediately | | | | | | |
| Extracting | 32.06 | 31.75 | 29.32 | 29.10 | 27.38 | 27.72 |
| with | 31.56 | | 28.82 | | 27.83 | |
| preservative | 31.62 | | 29.16 | | 27.94 | |
| immediately | | | | | | |
| 3 days | 31.1 | 31.28 | 30.85 | 30.95 | 29.40 | 29.09 |
| | 30.87 | | 31.08 | | 28.75 | |
| | 31.88 | | 30.91 | | 29.12 | |
| 7 days | 33.05 | 33.26 | 31.09 | 31.26 | 26.12 | 26.16 |
| | 33.23 | | 31.12 | | 25.87 | |
| | 33.51 | | 31.58 | | 26.49 | |

The performance test results of the nucleic acid preservation solution prepared in embodiment 2
(1) Human immune cells were added to the nucleic acid storage solution prepared in Example 2 and stored at room temperature for 1, 3, 7, and 14 days. After RNA was extracted, the purity and concentration were determined. The experiment was repeated 3 times. The results are shown in Table 10.

TABLE 10
RNA purity and concentration

| Days of | | Purity | | Concentration | |
|---|---|---|---|---|---|
| preservation | | (A260/A280) | Mean | (ng/μL) | Mean |
| Extracting | | 1.952 | 1.930 | 175.28 | 176.32 |
| without | | 1.905 | | 180.54 | |
| preservative | | 1.934 | | 173.15 | |
| immediately | | | | | |
| 1 | day | 1.887 | 1.890 | 158.52 | 153.52 |
| | | 1.864 | | 141.4 | |
| | | 1.92 | | 160.65 | |
| 3 | days | 1.992 | 1.956 | 118.4 | 111.83 |
| | | 1.891 | | 113.67 | |
| | | 1.985 | | 103.42 | |
| 7 | days | 1.862 | 1.906 | 99.41 | 90.43 |
| | | 1.952 | | 88.67 | |
| | | 1.903 | | 83.2 | |
| 14 | days | 1.992 | 1.942 | 62.04 | 64.88 |
| | | 1.92 | | 60.16 | |
| | | 1.915 | | 72.44 | |

(2) Human immune cells were added to the nucleic acid storage solution prepared in Example 2 and stored at room temperature for 1, 3, 7, and 14 days, and RNA was extracted for qPCR quantitative analysis. The experiment was repeated 3 times. The results are shown in Table 11.

TABLE 11
Ct value in qPCR result of RNA

| Days of | | |
|---|---|---|
| preservation | Ct Value | Mean |
| Extracting without | 20.43 | 20.80 |
| preservative | 20.89 | |
| immediately | 21.08 | |

| | | | |
|---|---|---|---|
| 1 | day | 23.48 | 23.27 |
| | | 23.11 | |
| | | 23.22 | |
| 3 | days | 22.98 | 23.30 |
| | | 23.39 | |
| | | 23.53 | |
| 7 | days | 23.52 | 23.41 |
| | | 23.79 | |
| | | 22.92 | |
| 14 | days | 23.31 | 23.03 |
| | | 22.94 | |
| | | 22.83 | |

(3) The accelerated stability results are shown in Table 12. Specifically, the nucleic acid storage solution prepared in Example 2 was placed at different temperatures and added to the RNA extracted from human immune cells, and measured its purity, concentration, and integrity.

TABLE 12
Result of accelerated stability

| | | | | | | | Number | |
|---|---|---|---|---|---|---|---|---|
| | | | | | Purity | Concentration | of bands | Clarity |
| Temperature | | Days | | pH | (A260/A280) | (ng/μL) | (28S:18S) | of band |
| Control | | no | | 4.51 | 1.925 | 133.51 | 2 | ✓ |
| 4° | C. | 1 | d | 4.62 | 1.925 | 112.42 | 2 | ✓ |
| | | 3 | d | 4.56 | 1.903 | 126.90 | 2 | ✓ |
| | | 7 | d | 4.50 | 1.864 | 115.53 | 2 | ✓ |
| | | 14 | d | 4.58 | 1.925 | 114.92 | 2 | ✓ |
| | | 28 | d | 4.52 | 1.841 | 98.60 | 2 | ✓ |
| 16° | C. | 1 | d | 4.61 | 1.905 | 128.36 | 2 | ✓ |
| | | 3 | d | 4.43 | 1.865 | 116.50 | 2 | ✓ |
| | | 7 | d | 4.58 | 1.915 | 99.34 | 2 | ✓ |
| | | 14d | d | 4.56 | 1.952 | 93.69 | 2 | ✓ |
| | | 28 | d | 4.44 | 1.925 | 78.80 | 2 | ✓ |
| 25° | C. | 1 | d | 4.56 | 1.896 | 135.62 | 2 | ✓ |
| | | 3 | d | 4.47 | 1.896 | 120.68 | 2 | ✓ |
| | | 7 | d | 4.45 | 1.902 | 108.70 | 2 | ✓ |
| | | 14 | d | 4.54 | 1.86 | 93.51 | 2 | ✓ |
| | | 28 | d | 4.53 | 1.852 | 79.34 | 2 | ✓ |
| 37° | C. | 1 d | d | 4.50 | 1.865 | 127.34 | 2 | ✓ |
| | | 3 | d | 4.55 | 1.852 | 108.33 | 2 | ✓ |
| | | 7 | d | 4.54 | 1.954 | 102.48 | 2 | ✓ |
| | | 14 | d | 4.53 | 1.896 | 95.90 | 2 | ✓ |
| | | 28 | d | 4.54 | 1.858 | 80.32 | 2 | ✓ |
| 45° | C. | 1 | d | 4.52 | 1.904 | 118.34 | 2 | ✓ |

| | | | | | | |
|---|---|---|---|---|---|---|
| 3 | d | 4.41 | 1.865 | 102.58 | 2 | ✓ |
| 7 | d | 4.62 | 1.903 | 94.20 | 2 | ✓ |
| 14 | d | 4.56 | 1.835 | 85.24 | 2 | ✓ |
| 28 | d | 4.49 | 1.824 | 69.38 | 2 | ✓ |

(4) The real-time stability results of the three batches of preservation solutions are shown in Table 13.1, Table 13.2, and Table 13.3. Specifically, the nucleic acid storage solution prepared in Example 2 was stored at room temperature for 1, 2, 3, 6, 9, 12, 18, 24, and 25 months. After adding human immune cells, the RNA extracted after adding human immune cells was tested for purity, concentration and integrity.

TABLE 13.1
The real-time stability results of the first batch

| | | | | | | | Number | |
|---|---|---|---|---|---|---|---|---|
| | | | | | Purity | Concentration | of bands | Clarity |
| Group | Set Time | | pH | Precipitation | (A260/A280) | (ng/μL) | (28S:18S) | of band |
| control | no | | 4.54 | no | 1.864 | 110.68 | 2 | ✓ |
| 25° C. | 1 | month | 4.63 | no | 1.887 | 91.08 | 2 | ✓ |
| | 2 | months | 4.51 | no | 1.887 | 92.65 | 2 | ✓ |
| | 3 | months | 4.55 | no | 1.92 | 105.08 | 2 | ✓ |
| | 6 | months | 4.64 | no | 1.903 | 94.23 | 2 | ✓ |
| | 9 | months | 4.53 | no | 1.841 | 88.22 | 2 | ✓ |
| | 12 | months | 4.54 | no | 1.905 | 92.16 | 2 | ✓ |
| | 18 | months | 4.52 | no | 1.841 | 91.37 | 2 | ✓ |
| | 24 | months | 4.57 | no | 1.831 | 92.50 | 2 | ✓ |
| | 25 | months | 4.57 | no | 1.992 | 87.18 | 2 | ✓ |

TABLE 13.2
The real-time stability results of the second batch

| | | | | | | | Number | |
|---|---|---|---|---|---|---|---|---|
| | | | | | Purity | Concentration | of bands | Clarity |
| Group | Set Time | | pH | Precipitation | (A260/A280) | (ng/μL) | (28S:18S) | of band |
| control | no | | 4.57 | no | 1.865 | 131.35 | 2 | ✓ |
| 25° C. | 1 | month | 4.52 | no | 1.852 | 128.52 | 2 | ✓ |
| | 2 | months | 4.60 | no | 1.954 | 115.20 | 2 | ✓ |
| | 3 | months | 4.52 | no | 1.896 | 105.28 | 2 | ✓ |
| | 6 | months | 4.57 | no | 1.858 | 104.05 | 2 | ✓ |
| | 9 | months | 4.48 | no | 1.904 | 98.20 | 2 | ✓ |
| | 12 | months | 4.54 | no | 1.865 | 122.16 | 2 | ✓ |
| | 18 | months | 4.49 | no | 1.903 | 111.35 | 2 | ✓ |
| | 24 | months | 4.59 | no | 1.835 | 90.50 | 2 | ✓ |
| | 25 | months | 4.46 | no | 1.824 | 80.13 | 2 | ✓ |

TABLE 13.3
The real-time stability results of the third batch

| | | | | | | | Number | |
|---|---|---|---|---|---|---|---|---|
| | | | | | Purity | Concentration | of bands | Clarity |
| Group | Set Time | | pH | Precipitation | (A260/A280) | (ng/μL) | (28S:18S) | of band |
| contro1 | no | | 4.52 | no | 1.827 | 124.36 | 2 | ✓ |
| 25° C. | 1 | month | 4.54 | no | 1.827 | 115.61 | 2 | ✓ |
| | 2 | months | 4.54 | no | 1.905 | 110.36 | 2 | ✓ |
| | 3 | months | 4.68 | no | 1.862 | 109.51 | 2 | ✓ |
| | 6 | months | 4.52 | no | 1.825 | 101.00 | 2 | ✓ |
| | 9 | months | 452 | no | 1.862 | 97.20 | 2 | ✓ |
| | 12 | months | 4.51 | no | 1.864 | 85.16 | 2 | ✓ |
| | 18 | months | 4.63 | no | 1.896 | 87.35 | 2 | ✓ |
| | 24 | months | 4.57 | no | 1.865 | 80.50 | 2 | ✓ |
| | 25 | months | 4.62 | no | 1.887 | 78.15 | 2 | ✓ |

(5) The results of the temperature influence in the transportation stability are shown in Table 14. Specifically, the nucleic acid storage solution prepared in Example 2 is packaged at different temperatures and different temperature and humidity conditions.

TABLE 14
The results of the temperature influence
in the transportation stability

| | Temperature | Humidity | Time | | |
|---|---|---|---|---|---|
| Test order | (° C.) | (% R.H) | (hr) | pH | Breakage |
| Humiture | 25.5 | 59 | 6 | 4.51 | no |
| pretreatment | | | | | |
| (lab humiture) | | | | | |
| Humiture | 40 | 90 | 72 | 4.55 | no |
| treatment | | | | | |
| (controllable | | | | | |
| humiture) | | | | | |

(6) The results of pressure influence in transportation stability are shown in Table 15, specifically the integrity of packaging of the nucleic acid storage solution prepared in Example 2 under different pressures.

TABLE 15
The results of pressure influence in transportation stability

| | Altitude | Altitude | | Torr | | | Duration | | |
|---|---|---|---|---|---|---|---|---|---|
| Group | (m) | (ft) | pH | (mm HG) | In.HG | kPa | (min) | pH | Breakage |
| Low pressure | 4267 | 14000 | 6.08 | 446.33 | 17.57 | 59.5 | 60 | 5.22 | no |

(7) The results of shock effects in transportation stability are shown in Table 16, specifically, the nucleic acid preservation solution prepared in Example 2 is packaged in completeness at different frequencies.

TABLE 16
The results of shock effects in transportation stability

| | | | | Surface | |
|---|---|---|---|---|---|
| | Shock spectrum | | | touching the | |
| | | PSD | | shock table | |
| | Frequency | grade | Time | (surface | |
| Model | (Hz) | (g2/Hz) | (min) | number) | Breakage |
| Random | 1 | 0.0001 | 30 | 3 | no |
| shock | 4 | 0.01 | 10 | 1 | no |
| | 100 | 0.01 | 10 | 4 | no |
| | 200 | 0.001 | 10 | 6 | no |

(8) The results of the impact of falling in the transportation stability are shown in Table 17, specifically the integrity of packaging of the nucleic acid storage solution prepared in Example 2 in different directions.

TABLE 17
The results of the impact of the drop in the transportation stability

| | Height of | | | |
|---|---|---|---|---|
| Number | drops | | | |
| of drops | (Inches) | Direction | | Breakage |
| 1 | 32 | angle | the weakest corner of the 3 | no |
| | | | sides, if not sure, test the | |
| | | | comer 2-3-5 | |
| 2 | 32 | edge | the shortest edge of the | no |
| | | | drop angle | |
| 3 | 32 | edge | the second longest edge of | no |
| | | | the drop angle | |
| 4 | 32 | edge | the longest edge of the | no |
| | | | drop angle | |
| 5 | 32 | surface | any smallest surface | no |
| 6 | 32 | surface | another smallest surface | no |
| 7 | 32 | surface | any medium surface | no |
| 8 | 32 | surface | another medium surface | no |
| 9 | 32 | surface | any largest surface | no |
| 10 | 32 | surface | another largest surface | no |

The performance test results of the nucleic acid preservation solution prepared in embodiment 3
(1) Human immune cells were added to the nucleic acid storage solution prepared in Example 3 and stored at room temperature for 1, 3, 7, and 14 days. After RNA was extracted, the purity and concentration were determined. The experiment was repeated 3 times. The results are shown in Table 18.

TABLE 18
RNA purity and concentration

| Days of | | Purity | | Concentration | |
|---|---|---|---|---|---|
| preservation | | (A260/A280) | Mean | (ng/μL) | Mean |
| Extracting | | 1.925 | 1.917 | 185.28 | 179.42 |
| without | | 1.902 | | 172.64 | |
| preservative | | 1.924 | | 180.35 | |
| immediately | | | | | |
| 1 | day | 1.865 | 1.886 | 128.52 | 120.13 |
| | | 1.903 | | 111.42 | |
| | | 1.891 | | 120.44 | |
| 3 | days | 1.862 | 1.913 | 105.47 | 102.52 |
| | | 1.952 | | 103.65 | |
| | | 1.925 | | 98.45 | |
| 7 | days | 1.858 | 1.901 | 99.41 | 90.45 |
| | | 1.925 | | 88.67 | |
| | | 1.92 | | 83.26 | |
| 14 | days | 1.936 | 1.919 | 72.04 | 71.03 |
| | | 1.905 | | 68.56 | |
| | | 1.915 | | 72.48 | |

(2) After human immune cells were added to the nucleic acid storage solution prepared in Example 3 and stored at room temperature for 1, 3, 7 or 14 days, RNA was extracted for qPCR quantitative analysis, and the experiment was repeated 3 times. The results are shown in Table 19.

Table 19 Ct value in qPCR result of RNA

| Days of preservation | Ct Value | Mean |
|---|---|---|
| Extracting without | 21.47 | 21.62 |
| preservative | 21.36 | |
| immediately | 22.04 | |

| | | | |
|---|---|---|---|
| 1 | day | 23.56 | 23.33 |
| | | 23.17 | |
| | | 23.25 | |
| 3 | days | 23.52 | 23.21 |
| | | 22.97 | |
| | | 23.15 | |
| 7 | days | 23.52 | 23.39 |
| | | 23.73 | |
| | | 22.92 | |
| 14 | days | 23.11 | 23.19 |
| | | 23.19 | |
| | | 23.26 | |

(3) The accelerated stability results are shown in Table 20. Specifically, the nucleic acid storage solution prepared in Example 3 was placed at different temperatures and added to the RNA extracted from human immune cells, and measured the purity, concentration, and integrity of the RNA.

TABLE 20
Result of accelerated stability

| | | | | | | | Number | |
|---|---|---|---|---|---|---|---|---|
| | | | | | Purity | Concentration | of bands | Clarity |
| Temperature | | Days | | pH | (A260/A280) | (ng/μL) | (28S:18S) | of band |
| Control | | no | | 5.97 | 1.925 | 125.52 | 2 | ✓ |
| 4° | C. | 1 | d | 6.02 | 1.858 | 102.42 | 2 | ✓ |
| | | 3 | d | 6.01 | 1.925 | 96.90 | 2 | ✓ |
| | | 7 | d | 5.98 | 1.92 | 90.55 | 2 | ✓ |
| | | 14 | d | 5.91 | 1.936 | 84.93 | 2 | ✓ |
| | | 28 | d | 6.05 | 1.905 | 88.68 | 2 | ✓ |
| 16° | C. | 1 | d | 6.06 | 1.915 | 108.36 | 2 | ✓ |
| | | 3 | d | 5.93 | 1.896 | 96.56 | 2 | ✓ |
| | | 7 | d | 5.97 | 1.864 | 99.44 | 2 | ✓ |
| | | 14 | d | 6.08 | 1.864 | 93.69 | 2 | ✓ |
| | | 28 | d | 5.94 | 1.887 | 88.76 | 2 | ✓ |
| 25° | C. | 1 | d | 5.95 | 1.887 | 105.69 | 2 | ✓ |
| | | 3 | d | 6.03 | 1.92 | 100.68 | 2 | ✓ |
| | | 7 | d | 5.99 | 1.903 | 98.75 | 2 | ✓ |
| | | 14 | d | 6.07 | 1.841 | 93.51 | 2 | ✓ |
| | | 28 | d | 5.96 | 1.905 | 89.37 | 2 | ✓ |
| 37° | C. | 1 | d | 6.10 | 1.841 | 107.34 | 2 | ✓ |
| | | 3 | d | 6.04 | 1.831 | 98.96 | 2 | ✓ |
| | | 7 | d | 6.09 | 1.992 | 92.25 | 2 | ✓ |
| | | 14 | d | 6.11 | 1.992 | 85.56 | 2 | ✓ |
| | | 28 | d | 5.92 | 1.925 | 90.32 | 2 | ✓ |
| 45° | C. | 1 | d | 6.00 | 1.856 | 108.37 | 2 | ✓ |

| | | | | | | |
|---|---|---|---|---|---|---|
| 3 | d | 5.98 | 1.925 | 102.58 | 2 | ✓ |
| 7 | d | 6.02 | 1.921 | 94.23 | 2 | ✓ |
| 14 | d | 6.11 | 1.926 | 95.54 | 2 | ✓ |
| 28 | d | 5.95 | 1.945 | 89.85 | 2 | ✓ |

(4) The real-time stability results of the three batches of storage solutions are shown in Table 21.1, Table 21.2, and Table 21.3. Specifically, the nucleic acid storage solution prepared in Example 3 is stored at room temperature 1, 2, 3, 6, 9, 12, 18. The purity, concentration and integrity of RNA extracted after adding human immune cells 24 and 25 months later were measured.

TABLE 21.1
The real-time stability results of the first batch

| | | | | | | | Number | |
|---|---|---|---|---|---|---|---|---|
| | | | | | Purity | Concentration | of bands | Clarity |
| Group | Set Time | | pH | Precipitation | (A260/A280) | (ng/μL) | (28S:18S) | of band |
| control | no | | 5.99 | no | 1.827 | 105.68 | 2 | ✓ |
| 25° C. | 1 | month | 5.98 | no | 1.905 | 92.08 | 2 | ✓ |
| | 2 | months | 6.01 | no | 1.827 | 92.41 | 2 | ✓ |
| | 3 | months | 5.95 | no | 1.864 | 85.68 | 2 | ✓ |
| | 6 | months | 5.94 | no | 1.86 | 84.25 | 2 | ✓ |
| | 9 | months | 5.97 | no | 1.903 | 88.85 | 2 | ✓ |
| | 12 | months | 6.06 | no | 1.896 | 92.16 | 2 | ✓ |
| | 18 | months | 6.11 | no | 1.992 | 81.45 | 2 | ✓ |
| | 24 | months | 5.91 | no | 1.992 | 82.20 | 2 | ✓ |
| | 25 | months | 5.96 | no | 1.925 | 85.36 | 2 | ✓ |

TABLE 21.2
The real-time stability results of the second batch

| | | | | | | | Number | |
|---|---|---|---|---|---|---|---|---|
| | | | | | Purity | Concentration | of bands | Clarity |
| Group | Set Time | | pH | Precipitation | (A260/A280) | (ng/μL) | (28S:18S) | of band |
| control | no | | 6.03 | no | 1.936 | 101.82 | 2 | ✓ |
| 25° C. | 1 | month | 5.95 | no | 1.887 | 98.52 | 2 | ✓ |
| | 2 | months | 6.11 | no | 1.865 | 95.45 | 2 | ✓ |
| | 3 | months | 5.97 | no | 1.872 | 93.52 | 2 | ✓ |
| | 6 | months | 5.99 | no | 1.862 | 94.05 | 2 | ✓ |
| | 9 | months | 5.98 | no | 1.903 | 92.75 | 2 | ✓ |
| | 12 | months | 5.94 | no | 1.862 | 89.44 | 2 | ✓ |
| | 18 | months | 6.07 | no | 1.852 | 81.38 | 2 | ✓ |
| | 24 | months | 6.09 | no | 1.925 | 85.77 | 2 | ✓ |
| | 25 | months | 5.92 | no | 1.835 | 82.65 | 2 | ✓ |

TABLE 21.3
The real-time stability results of the third batch

| | | | | | | | Number | |
|---|---|---|---|---|---|---|---|---|
| | | | | | Purity | Concentration | of bands | Clarity |
| Group | Set Time | | pH | Precipitation | (A260/A280) | (ng/μL) | (28S:18S) | of band |
| control | no | | 6.05 | no | 1.925 | 108.25 | 2 | ✓ |
| 25° C. | 1 | month | 6.04 | no | 1.981 | 102.79 | 2 | ✓ |
| | 2 | months | 5.96 | no | 1.862 | 100.32 | 2 | ✓ |
| | 3 | months | 5.98 | no | 1.904 | 99.85 | 2 | ✓ |
| | 6 | months | 6.10 | no | 1.858 | 91.36 | 2 | ✓ |
| | 9 | months | 6.07 | no | 1.915 | 97.34 | 2 | ✓ |
| | 12 | months | 5.97 | no | 1.887 | 95.16 | 2 | ✓ |
| | 18 | months | 6.02 | no | 1.952 | 94.35 | 2 | ✓ |
| | 24 | months | 6.11 | no | 1.824 | 80.50 | 2 | ✓ |
| | 25 | months | 5.91 | no | 1.841 | 78.41 | 2 | ✓ |

(5) The results of temperature influence in transportation stability are shown in Table 22. Specifically, the packaging integrity of the nucleic acid storage solution prepared in embodiment 3 is at different temperatures and humidity.

TABLE 22
The results of the temperature influence
in the transportation stability

| | Temperature | Humidity | Time | | |
|---|---|---|---|---|---|
| Test order | (° C.) | (% R.H) | (hr) | pH | Breakage |
| Humiture | 25.5 | 59 | 6 | 6.03 | no |
| pretreatment | | | | | |
| (lab humiture) | | | | | |
| Humiture | 40 | 90 | 72 | 6.01 | no |
| treatment | | | | | |
| (controllable | | | | | |
| humiture) | | | | | |

(6) The results of pressure influence in transportation stability are shown in Table 23, specifically the integrity of packaging of the nucleic acid storage solution prepared in Example 3 under different pressures.

TABLE 23
The results of pressure influence in transportation stability

| | Altitude | Altitude | | Torr | | | Duration | | |
|---|---|---|---|---|---|---|---|---|---|
| Group | (m) | (ft) | pH | (mm HG) | In.HG | kPa | (min) | pH | Breakage |
| Low pressure | 4267 | 14000 | 6.08 | 446.33 | 17.57 | 59.5 | 60 | 6.02 | no |

(7) The results of the impact of shock in the transportation stability are shown in Table 24, specifically the packaging integrity of the nucleic acid preservation solution prepared in embodiment 3 at different frequencies.

TABLE 24
The results of shock effects in transportation stability

| | | | | Surface | |
|---|---|---|---|---|---|
| | | | | touching the | |
| | Shock spectrum | | | shock table | |
| | Frequency | PSD grade | Time | (surface | |
| Model | (Hz) | (g2/Hz) | (min) | number) | Breakage |
| Random | 1 | 0.0001 | 30 | 3 | no |
| shock | 4 | 0.01 | 10 | 1 | no |
| | 100 | 0.01 | 10 | 4 | no |
| | 200 | 0.001 | 10 | 6 | no |

(8) The results of the impact of drop in transportation stability are shown in Table 25, specifically the integrity of the nucleic acid storage solution prepared in Example 3 in different directions.

TABLE 25
The results of the impact of the drop in the transportation stability

| | Hight of | | | |
|---|---|---|---|---|
| Number | drops | | | |
| of drops | (Inches) | Direction | | Breakage |
| 1 | 32 | angle | the weakest corner of the 3 | no |
| | | | sides, if not sure, test the | |
| | | | corner 2-3-5 | |
| 2 | 32 | edge | the shortest edge of the | no |
| | | | drop angle | |
| 3 | 32 | edge | the second longest edge of | no |
| | | | the drop angle | |
| 4 | 32 | edge | the longest edge of the | no |
| | | | drop angle | |
| 5 | 32 | surface | any smallest surface | no |
| 6 | 32 | surface | another smallest surface | no |
| 7 | 32 | surface | any medium surface | no |
| 8 | 32 | surface | another medium surface | no |
| 9 | 32 | surface | any largest surface | no |
| 10 | 32 | surface | another largest surface | no |

From the above results, it can be seen that under normal temperature conditions, the nucleic acid storage solution prepared in embodiments 1-3 can stably store the sample nucleic acid for 14 days and the preservation solution is within 25 months of validity. All performance indicators meet the quality requirements. After the humidity, pressure, vibration frequency, and drop direction tests, the product and packaging have no obvious damage. The nucleic acid preservation solution prepared in embodiment 1 shows the best effect.

The present invention does not need to ensure the survival of cells in the sample, but it only needs to ensure that stable and quality-guaranteed nucleic acids can be extracted from the preserved tissue sample.

The above description of the disclosed embodiments enables professional technicians in the field to realize or use the invention. Various modifications to these embodiments will be obvious to those skilled in the art, and the general principles defined herein can be implemented in other embodiments without departing from the spirit or scope of the present invention. Therefore, the present invention will not be limited to the embodiments shown in this document, but should conform to the widest scope consistent with the principles and novel features disclosed in this document.

1. A nucleic acid preservation solution, by weight percentage, comprising:
citric acid 13%-35%,
Tween 20 0.50%-2.50%,
disodium ethylene diamine tetraacetic acid (EDTA) 13%-40%,
sodium sulfate 35%-70%,
polyethylene glycol octyl phenyl ether 0.50%-2.50%, and
the balance being diethylpyrocarbonate (DEPC) water.

2. The nucleic acid preservation solution of claim 1, wherein the solution comprises, by weight percentage, citric acid 21%, Tween 20 1.50%, disodium EDTA 35.9%, sodium sulfate 40%, polyethylene glycol octyl phenyl ether 1.50%, and the balance being DEPC water.

3. The nucleic acid preservation solution of claim 1, wherein the pH of the preservation solution of the nucleic acid is 4.5-6.

4. A preparation method of a nucleic acid preservation solution, comprising steps of:
preparing the citric acid 13%-35%, Tween 20 0.50%-2.50%, disodium EDTA 13%-40%, sodium sulfate 35%-70% and polyethylene glycol octyl phenyl ether 0.50%-2.50% according to weight percentage, and dissolving in balance being diethylpyrocarbonate DEPC water to get dissolved solution;
regulating the pH of the dissolved solution to 4.5-6 with a pH meter;
sterilizing the regulated solution through a 0.22 μM water filtration membrane to obtain nucleic acid preservation solution.

5. The preparation method of the nucleic acid preservation solution of claim 4, wherein the method further comprises dispensing the prepared nucleic acid preservation solution aseptic and enzyme free into sampling tubes; the short-term storage and testing temperature of the sample tube is 0-50° C.; and the long-term storage temperature of the sample tube is 4° C.

6. An application of the nucleic acid preservation solution of claim 1 in the preservation and transportation of biological samples, wherein the preservation and transportation temperature is 0-50° C.

7. The application of the nucleic acid preservation solution in the preservation and transportation of biological samples of claim 6, wherein the preservation and transportation temperature is 25° C.

8. The application of the nucleic acid preservation solution of claim 6 in the preservation and transportation of biological samples, wherein the biological samples are human mammal blood, urine, feces, sputum, saliva and throat swab collection fluid.

9. The application of the nucleic acid preservation solution of claim 6 in the preservation and transportation of biological samples, wherein the solution is used for storing coronavirus nucleic acid.
